(19) United States
(12) Patent Application Publication
(10) Pub. No.: US 2024/0000649 A1
(43) Pub. Date: Jan. 4, 2024

(54) INTERCONNECTED BRACE AND CRUTCH SYSTEM

(71) Applicant: Medos International Sàrl, Le Locle (CH)

(72) Inventors: David B. Spenciner, North Attleboro, MA (US); Cheng-Ju Wu, Raynham, MA (US); Drew Miller, Raynham, MA (US); Steven Nguyen, Raynham, MA (US); Ravi Patel, Raynham, MA (US)

(21) Appl. No.: 17/855,715

(22) Filed: Jun. 30, 2022

(51) Int. Cl. A61H 3/02; A61F 5/01; A61N 1/04; G16H 40/67

(52) U.S. Cl. CPC A61H 3/02; A61F 5/0109; A61N 1/0452; G16H 40/67; A61H 2201/5015; A61H 2201/5064; A61H 2201/5058; A61H 2201/5069; A61H 2201/5061; A61H 2230/62

(57) ABSTRACT

A patient brace system and method of using the same are disclosed for treatment of lower extremity injuries. Described brace systems include a crutch, at least one crutch sensor, a patient brace, at least one controller, and a rehabilitation application. The crutch sensor(s) measure one or more crutch parameters. The controller(s) are operable to receive and process a signal(s) indicative of the crutch parameter(s) and produces patient gait data that, in turn, is communicated to a data hub. In some embodiments, the patient brace may also include at least one brace sensor configured to similarly communicate with the data hub. The rehabilitation application is communication with the data hub and determines a response output based on the information fed to the data hub, the application then communicating the response output to at least one of a caregiver, a user of the patient brace, and/or to the patient brace.

### FIELD

The present disclosure relates to systems, devices, and methods for using a brace system to aid in the treatment and recovery process after trauma, and more particularly relates to using a brace that communicates with other medical components, such as a crutch and/or related “app,” to allow for more informed adjustment of the brace as part of a patient recovery protocol.

### BACKGROUND

Patients may suffer from disease or trauma to a body part, necessitating the performance of a surgery for treatment. If a surgery is indicated, the surgical procedure may require a prolonged period of rehabilitation and recovery. Alternatively, in some instances, the patient may need to be relieved from pain by limiting impact on the affected region, regardless of whether surgery is involved. For example, a patient requiring the use of a crutch and/or brace due to a sprain or break of an ankle or knee. One factor that impacts this rehabilitation and recovery process is patient adherence to protocols that require limited use of the body part that has undergone the surgery. The rehabilitation process limiting body use is especially critical when it affects the patient's mobility. The patient may use a weight bearing device to ambulate and/or use a brace to shield the injured limb from use and impact.

Though rehabilitation protocols may vary, the goal of most protocols includes taking the patient from a state of limited weight bearing on the injured limb to a state of full weight bearing on the injured limb. Many protocols will progress through various phases of increasing partial weight bearing during the healing process. Too much weight too soon can cause further injury. Too little weight can compromise healing because mechanical stress is needed to rebuild muscles and to stimulate bone growth. Some non-limiting locations in the body where a surgery may necessitate this prolonged period of rehabilitation and recovery include regions of the lower extremity such as the knee, the ankle, and the foot.

Braces are estimated to be used by over a million people annually and are a common prescribed orthotic aids. Assistive walking devices, such as crutches, canes, and walkers, are also used by many patients temporarily during the rehabilitation and recovery phase. Traditionally, the patient uses a brace in coordination with a walking device to ensure rehabilitation and recovery of the knee, ankle, or foot. The walking device helps prevent undesired amounts of force to be applied to the recovering body part(s) during movement (e.g., walking) by the patient. A patient may use what may sometimes be referred to as a “smart brace” to aid in the rehabilitation and recovery process. It may be considered “smart” because it may be fitted with a motion sensor that communicates with an application, or “app.” Though such smart braces may provide useful information about the muscles they are protecting, there is usually insufficient real estate on such braces to provide sensors capable of obtaining a more diverse array of information that can be useful in helping a patient's recovery, as well as to have associated with the brace sufficient processing, power, and charging capabilities. Additionally, existing smart braces are not typically robust enough to easily withstand routine or significant exposure to moisture though sweat, rain, or spillage. These limitations affect the choice of technology incorporated in the brace.

Another challenge that exists with much of existing brace technology is that it is more of a “one size fits all” type of device. Braces are not often individually tailored to a specific user. Braces can be off-the-shelf solutions, for example, coming in just a few different sizes. Further, they typically cannot be easily adjusted, for example, by a user. Typically brace adjustments occur during a patient visit with a caregiver (e.g., doctor, physical therapist, clinician) and thus the brace stays in the same configuration between visits, allowing for a static, rather than a dynamic, recovery device. As a result, the recovery tied to the brace cannot be dynamically adjusted to account for individual patient behavior and response. Each patient responds to therapy and rehabilitation in a unique manner, and thus, it can be beneficial, and in some instances necessary, to change the rehabilitation protocol to reflect the patient's response. However, most changes to the rehabilitation protocol are not made instantaneously and depend on caregiver input. In some cases the patient may not interact with the caregiver in a timely manner to determine an effective change to the rehabilitation protocol. Furthermore, in many such cases, it can be beneficial, and in some instances necessary, to change the functioning of the brace or the crutch automatically or manually in real-time to reflect the patient's need.

Accordingly, there is a need for improved systems and methods that allow for braces, or brace adjustments, to be made dynamically, in response to data gathered by the brace, and/or systems, devices, and/or sensors associated with the brace.

### SUMMARY

The systems, devices, and methods described herein generally relate to using data generated by one or more components in communication with a medical treatment device (e.g., a brace) to inform or cause adjustments to be made to the medical treatment device to aid in a patient's treatment and recovery process. More particularly, the present disclosure primarily provides for the medical treatment device to be a brace, and thus the provided system, referred to herein as a brace system, includes a brace, a walking device (e.g., a crutch(es), cane, or walker), and an app that can communicate with each other to exchange information relative to the treatment and recovery of the patient. More generally, the walking device can be referred to as a rehabilitation aid, as it does not necessarily have to be a walking device, such aid depending on the type of injury being treated. Additionally, the brace and/or walking device can include sensors and the like that can be used to collect data and communicate the data to the various components of the system. Further, in at least some embodiments, the brace and/or walking device can include one or more actuators or the like that can provide for adjustments to the brace and/or walking device in response to the collected data and any instructions resulting therefrom. Such adjustments can be mechanical or otherwise and allow for the adjustment of a rehabilitation protocol being implemented for patient recovery in a more informed and/or timelier manner. A person skilled in the art will appreciate that such systems can be implemented with other medical treatment devices in lieu of or in addition to braces, including but not limited to slings, casts, flexible sleeves, gloves, splints, plasters, skin tapes (e.g., kinesiotape), and/or soft or hard boots.

One embodiment of a brace system in accordance with the present application includes a crutch, at least one crutch sensor configured to measure one or more parameters associated with the crutch and generate crutch signals in view of the same, a controller, a patient brace, and a rehabilitation application. The controller is operable to receive the generated crutch signals from the at least one crutch sensor, and is further operable to process the generated crutch signals to produce patient gait data and communicate at least one of the one or more parameters measured by the at least one crutch sensor or the patient gait data to a data hub. The rehabilitation application is in communication with the data hub and is configured to receive a clinical input. The rehabilitation application is further configured to process the clinical input and the patient gait data to determine a response output and communicate the response output to at least one of a caregiver, a user of the patient brace, or to the patient brace.

The devices and methods described herein can have a number of additional features and/or variations, all of which are within the scope of the present disclosure. For example, the system can include a second controller that can be operable to receive at least one of one or more parameters measured by the at least one crutch sensor or the patient gait data. The second controller can be further operable to process at least one of the one or more parameters measured by the at least one crutch sensor and/or the patient gait data and determine a load acting on at least one of the patient brace or patient knee. By way of further example, the patient brace system can further include at least one brace sensor and a second controller. The brace sensor(s) can be configured to measure one or more parameters associated with the brace and generate brace signals in view of the same. The second controller can be operable to receive the generated brace signals from the at least one brace sensor and can be further operable to process the generated brace signals to produce patient brace data and communicate at least one of the one or more parameters measured by the at least one brace sensor or the patient brace data to the data hub. The rehabilitation application can be further configured to process the patient brace data in conjunction with determining the response output.

The at least one crutch sensor and the controller of the patient brace system can be located on the crutch and/or the at least one brace sensor and the second controller can be located on the brace of the patient brace system. In some such embodiments, the controller and the second controller can be configured to transmit the patient gait data and the patient brace data, respectively, to each other. In some embodiments, the second controller can be operable to receive the output response generated by the rehabilitation application, and can be further operable to process the generated output response. In some such embodiments, the brace can respond to the output response.

The patient gait data and patient brace data can include, for example, one or more of information, electrons, energy, and/or signals. In some embodiments, the at least one brace sensor can be operable to determine movement and/or location of the patient brace in relation to the crutch. The patient brace data can include, for example, at least one of: knee angle, knee location, patient posture, muscle tone, swing time, and/or toe touch force data.

In some embodiments, the brace can include at least one actuator and/or at least one electrode, either or both being configured to stimulate at least one of a patient muscle, change patient brace stiffness, and/or change freedom of patient brace movement. In some embodiments, the patient gait data can include ground reaction force and/or gait timing. The response output can be passive and can include at least one of an audible indication, a visual indication, and/or a tactile indication to a user. Alternatively, the response output can be active and can occur in real time.

The data hub can be located on the crutch. The rehabilitation application can be located on an electronic device accessible to the user of the patient brace. In some embodiments, the crutch of the patient brace system can be a first crutch, and the patient brace system can further include a second crutch. The second crutch can be used as a standard known datum for calibration of patient gait data.

One embodiment of a method of adjusting a knee brace in accordance with the present disclosure includes determining patient gait data based on one or more parameters associated with a crutch and communicating the determined patient gait data to at least one of a controller or a data hub. The method further includes, based on the communicated patient gait data, performing at least one of the following actions: causing a knee brace to make an auto adjustment in response to the communicated patient gait data, or instructing at least one of a caregiver or a user of a knee brace to adjust the knee brace. A person skilled in the art will appreciate other medical and/or rehabilitation devices can be used in lieu of a knee brace and/or a crutch.

In some embodiments, the controller can be located on a knee brace, and the action of communicating the gait data can depend on a positional relationship between the knee brace and the crutch. The at least one crutch sensor can be configured to measure the one or more crutch parameters associated with the patient data gait. Further, the action of communicating the determined patient gait data to at least one of a controller or a data hub can further include operating the controller to process the one or more crutch parameters to produce the patient gait data.

The method can further include operating a second controller to process one or more brace parameters measured by at least one brace sensor on the knee brace to produce patient brace data. In some embodiments, causing the knee brace to make the auto adjustment can include operating at least one brace actuator and/or at least one electrode based on the patient gait data. In some embodiments causing the knee brace to make the auto adjustment can include using the patient gait data and the patient brace data in a load balancing algorithm. Alternatively, or additionally, the action of causing the knee brace to make the auto adjustment can include operating the at least one brace actuator and/or the at least one electrode to change at least one of a knee brace stiffness and/or a knee brace freedom of motion. In some embodiments, the action of causing the knee brace to make the auto adjustment can include operating the at least one brace actuator and/or the at least one electrode to lock the knee brace. Still further, in some embodiments, the action of causing the knee brace to make the auto adjustment can include operating at the at least one brace actuator and/or the at least one electrode to change a temperature associated with at least part of the knee brace. Causing the knee brace to make the auto adjustment can include transferring energy between the crutch and the knee brace.

A method for formulating a patient rehabilitation protocol in accordance with the present disclosure includes processing patient input from a data hub, processing clinical input from a caregiver, and assessing patient gait based on the patient input and the clinical input by using at least one of inverse kinematics or inverse dynamics. The method further includes at least one of providing or adjusting a patient rehabilitation protocol based on the patient gait. The patient input includes at least one of patient gait data or patient brace data.

In some embodiments, the patient gait can be limited, and the patient rehabilitation protocol can be determined to be aggressive with an increase in activity level. The patient rehabilitation protocol can include a sensory guide. In some such embodiments, the method can further include operating the sensory guide to determine which muscle or muscles need stimulation. The patient rehabilitation protocol can be accessible to a user by an application on at least one of a computer or a smart device. In some embodiments, the clinical input can include time since surgery and/or time in rehabilitation. The patient input can be processed in real time from the data hub. In some embodiments, providing or adjusting a patient rehabilitation protocol can further includes providing a user an exercise regimen and/or a rehabilitation plan.

Any of the features or variations described above can be applied to any particular aspect or embodiment of the present disclosure in a number of different combinations. The absence of explicit recitation of any particular combination is due solely to the avoidance of repetition in this summary.

### BRIEF DESCRIPTION OF DRAWINGS

This disclosure will be more fully understood from the following detailed description taken in conjunction with the accompanying drawings, in which:

FIG. 1 illustrates one embodiment of a brace system in accordance with the present disclosure;

FIG. 2 illustrates one embodiment of a brace including sensors and actuators;

FIG. 3 is a flowchart that shows one embodiment of the relationship between different components of an exemplary brace system in accordance with the present disclosure;

FIG. 4 is a screenshot of one embodiment of a user interface in a rehabilitation application used by a patient using a brace system in accordance with the present disclosure;

FIG. 5 is a flowchart that shows one embodiment of an algorithm used for determining or adjusting post-operative treatment following an ACL reconstruction procedure; and

FIG. 6 is a flowchart that shows one embodiment of an interaction between data collected by sensors in a brace system in accordance with the present disclosure, assessment(s) made by caregivers, information stored in clinical databases, and one or more algorithms operable in a rehabilitation application, the flowchart helping to determine changes to a patient's rehabilitation protocol in view of the data, assessment(s), information, and algorithm(s).

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure. Like-numbered components across embodiments generally have similar features unless otherwise stated or a person skilled in the art would appreciate differences based on the present disclosure and his/her knowledge. Accordingly, aspects and features of every embodiment may not be described with respect to each embodiment, but those aspects and features are applicable to the various embodiments unless statements or understandings are to the contrary.

The figures provided herein are not necessarily to scale, although a person skilled in the art will recognize instances where the figures are to scale and/or what a typical size is when the drawings are not to scale. While in some embodiments movement of one component is described with respect to another, a person skilled in the art will recognize that other movements are possible. To the extent features or steps are described herein as being a “first feature” or “first step,” or a “second feature” or “second step,” such numerical ordering is generally arbitrary, and thus such numbering can be interchangeable. Moreover, a person skilled in the art will appreciate that not all of the method steps disclosed herein are required, and, in view of the present disclosure, will understand how modifications can be made to each step, the order of the steps, the limitation of certain steps, etc. without departing from the spirit of the present disclosure while still achieve the desired goals. Additionally, a number of terms may be used throughout the disclosure interchangeably but will be understood by a person skilled in the art.

The present disclosure relates to systems, devices, and methods for treating an injury, such an injury to a leg, that requires the use of a medical treatment device, such as a brace, and a rehabilitation aid, such as a crutches (or a single crutch), cane, or walker. Such devices and aids can be in communication with each other and/or an app, with the app being able to gather data and/or, in some more sophisticated options, able to provide for actions to occur in response to the data collected by the devices and/or aids and/or data otherwise inputted into the app. The actions can include, by way of non-limiting example, directions to a user/wearer of the medical device and/or aid to make adjustments to the same, directions to a caregiver or the like to make adjustments to the medical device and/or aid, or automated directions that cause one or more changes to be made to the medical device and/or aid in response to directions from the app, such as one or more actuators being operated to make an adjustment to one or more parameters of the medical device and/or aid.

More particularly, embodiments of a brace system disclosed herein can aid in determining or adjusting a patient rehabilitation program used by the patient based on data collected or otherwise inputted into the system. The patient rehabilitation program may be a standalone rehabilitation program, or it may be pre- and/or post-operative. As the data is provided to the system, it can allow for the patient's rehabilitation program to more effectively account for that particular patient's response to the prescribed treatment. The patient may need a brace following some form of injury or trauma. The brace may be the main form of treatment, or it can be part of a treatment plan, such as being used before and/or after surgery to treat or support the patient's knee (e.g., knee brace), ankle (e.g., ankle brace), or foot (e.g., foot brace). While the characteristics of the different braces can differ, at least in part, based on the location, a person skilled in the art will appreciate the scope of the present disclosure is applicable to many types of braces and other medical treatment devices. Thus, to the extent the present disclosure uses a knee brace in illustrated embodiments, the use of knee brace is non-limiting, and other braces, including but not limited to an ankle brace a foot brace, or braces for other parts of the body not necessarily associated with a leg, can be used and/or otherwise adapted for use in conjunction with the features and techniques of the present disclosures. The brace may be a soft brace, an unloader brace, a rehabilitative brace, an external fixator frame, a soft cast, or a hard cast, among other types of medical treatment devices. The need of the brace system can be determined by a caregiver (e.g., doctor, nurse, aide, physical therapist, medical assistant, surgical assistant, nursing home assistant, or other medical personnel). A patient may use one or more walking devices, such as crutches, canes, or walkers, along with one or more braces to aid in mobility during treatment. As discussed in detail herein, the brace system used by the patient during the treatment period includes a brace that communicates with a crutch. It will be appreciated that references herein to a brace or braces is by no means limiting and that the disclosures related to the same are equally applicable to medical treatment devices more generally. Likewise, references herein to a crutch or crutches is by no means limiting, and that the disclosures related to the same are equally applicable to rehabilitation aids more generally. The foregoing two statements notwithstanding, a person skilled in the art will appreciate some differences that may be necessitated by virtue of the device or aid being of a different design and/or different purpose and such person would understand what adjustments are necessary to implement the disclosed features and methods in such alternative devices and aids.

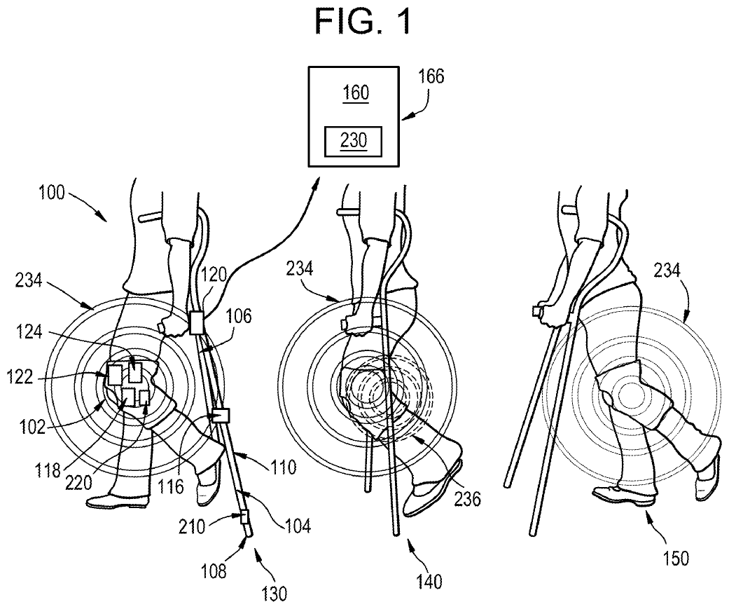

FIG. 1 illustrates one embodiment of a brace system 100 of the present disclosure with components for use by the patient. The brace system 100 includes a knee brace 102, a crutch 104, a data hub 120, and a rehabilitation application or “app” 230. The brace 102 is a medical treatment device used by the patient to protect the part of the body that is being treated. The crutch 104 is a walking device used by the patient to ambulate. One or both of the brace 102 and the crutch 104 can include sensors 118 and 116, respectively, to collect data, as discussed in greater detail below. In some embodiments, the brace system 100 may include a second crutch 106. The second crutch can be used in a similar manner as the crutch 104, and/or work in tandem with the crutch 104, and/or it can be used as a standard known datum in reference to an object in motion. With respect to the former, for example, both crutches 104, 106 can include sensors 116, with those sensors 116 being designed to gather similar data, different data, and/or data that is complementary between the two crutches and those sensors 116 being the same and/or different between the two crutches 104, 106. Further details about the sensors 116, as well as sensors 118 for the brace 102, are provided below. With respect to the latter, for example, the second crutch 106 can be used for data calibration and/or for better trajectory mapping of patient movement.

The brace 102 in the brace system 100 can include, or otherwise be associated with (e.g., have sensors that monitor the brace 102 but are not necessarily coupled to, disposed on, etc. the brace 102), one or more brace sensors 118, where otherwise associated with includes having sensors that monitor the braces 102 but are not necessarily coupled to, disposed on, etc. the brace 102. The one or more brace sensors 118 can be included near the brace, on the brace, inside the brace, woven into the brace, and/or be positioned with respect to the brace such that the sensor(s) can monitor one or more parameters associated with the brace 102. The brace sensors 118 can be configured to measure one or more brace parameters. Alternatively, or additionally the brace sensors 118, and/or other components of the brace system 100 and/or brace 102, can be configured to detect signals from other devices disposed in and/or otherwise associated with the body. For example, the brace sensors can be configured to detect signals from one or more implants located inside the patient. The brace sensors 118 can, after measuring various parameters and/or detecting various signals, generate signals based on those brace parameters and/or detected signals, the generated signals being used as the means by which the measured parameter(s) and/or detected signal(s) are communicated by the sensor(s) 118 to another component, location, etc. The brace parameters may indicate patient treatment or recovery parameters and/or brace properties, including but not limited to knee angle, swing time, patient posture, muscle tone, toe touch force data, brace charging status, brace stiffness, muscle firing amplitude or timing, step count, muscle or brace anatomic position, and/or anatomic motion (e.g., patellar tracking, varus or valgus position). The brace sensors 118 can include surface sensors, electromyography sensors (EMGs), vibration sensors, pressure sensors, position sensors, cameras, motion detectors, gyroscopes, load cells, force sensors, electric current sensors, and/or Hall Effect sensors.

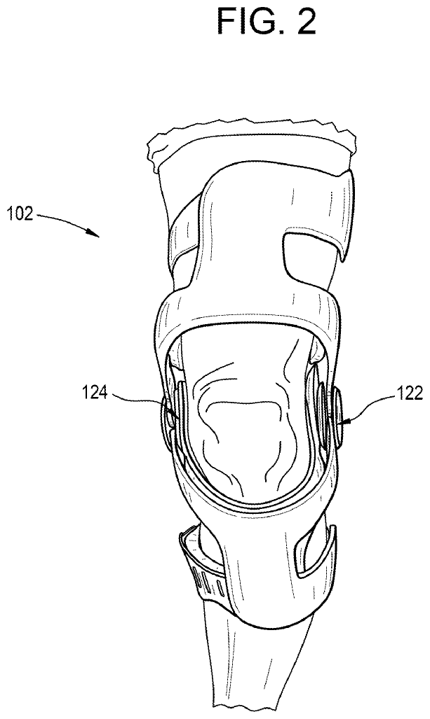

With reference to FIG. 2, the brace 102 can also include one or more actuators 122 and/or one or more electrodes 124 that are operable to respond to signals and/or instructions communicated to them. The actuators 122 of the brace 102 can be used to adjust the movement of the brace, for example by increasing or decreasing an angle of rotation permitted by the brace 102 and/or by increasing or decreasing an amount of force applied by the brace 102 to a body part (e.g., a knee) disposed in the brace 102. The actuators 122 can be wire actuators, shape memory alloy actuators, magnetic actuators, pneumatic actuators, hinges, elastomers, and/or springs. The actuators 122 or electrodes 124 can respond to mechanical signals and/or electrical signals, and can be manually or automatically activated. Manual activation can include, for example, a user or caregiver adjusting the actuator(s) based on feedback provided in view of the measured parameters. This may include, by way of non-limiting example, a person adjusting a degree of rotation permitted by the hinge of the brace 102 by adjusting one or more adjustment components (e.g., screws, though a person skilled in the art will appreciate many other options for achieving a similar purpose, including mechanical and/or electrical options) that affect a rotation range of the brace 102 (i.e., the limits of the range). Automatic activation can include, for example, the brace itself being adjusted via the actuator(s) based on feedback provided in view of the measured parameters. This may include, by way of non-limiting example, a person adjusting how tightly the brace 102 holds the body part (e.g., knee) by adjusting one or more adjustment components (e.g., screws, though again, a person skilled in the art will appreciate many other options for achieving a similar purpose, including mechanical and/or electrical options) that affect how much force the brace 102 applies to the body part. Alternatively or additionally, manual activation can include a person adjusting how tightly the brace 102 holds the body part (e.g., knee) by actively altering device dimensions and the like to affect how much force the brace 102 applies to the body part. Similarly, the electrodes 124 can be used, by way of non-limiting example, to adjust a temperature, stiffness, and/or compliance of the brace 102. The actuators 122 and/or electrodes 124 can be used to adjust, change, or redefine brace properties including, but not limited to brace stiffness, brace temperature, brace pressure, degree of motion, and/or motion limits.

Turning back to FIG. 1, the crutch 104 in the brace system 100 can include a crutch tip 108 coupled to a crutch body 110 and can include, or otherwise be associated with (e.g., have sensors that monitor the crutch 104 but are not necessarily coupled to, disposed on, etc. the crutch 104), one or more crutch sensors 116. The crutch sensors 116 can be included near the crutch, on the crutch, inside the crutch, and/or be positioned with respect to the crutch such that the sensor(s) can monitor one or more parameters associated with the crutch 104. Reference to a first component, such as the crutch sensor(s) 116, being “on” a second component, such as the crutch 104, of the system 100 can include the first component being located anywhere on and/or in the second component. The crutch sensors 116 can be configured to measure one or more crutch parameters and generate signals based on those crutch parameters, the signals being used as the means by which the measured parameter(s) are communicated by the sensor(s) 116 to another component, location, etc. The crutch parameters may indicate patient treatment or recovery parameters and/or crutch properties, including but not limited to patient gait phase event data, patient gait timing data, patient joint movement data, ground reaction force, crutch charging status, and/or crutch length. The crutch sensors 116 can include a motion detector, vibration sensor, pressure sensor, accelerometer, gyroscope, microphone, and/or magnetometer. By way of non-limiting example, a crutch sensor 116 located on the crutch tip 108 can be configured to measure the force and/or orientation of the crutch tip 108. In other embodiments, the crutch sensors 116 can be located on the crutch body 110 instead of, or in addition to, being located on the crutch tip 108.

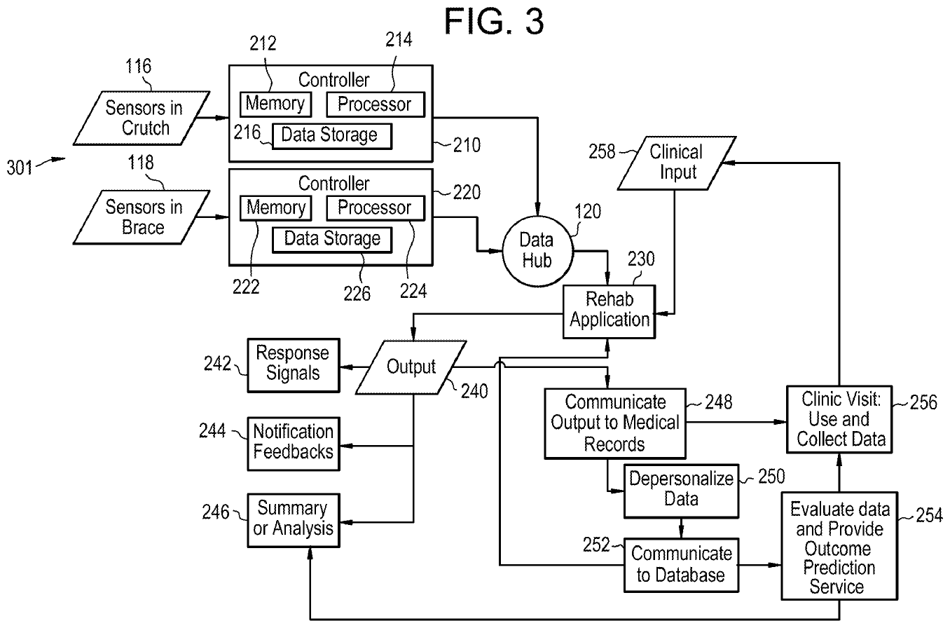

As shown in FIGS. 1 and 3, the crutch 104 can include a controller 210 disposed on, located inside, coupled to, and/or otherwise associated with it. In alternative embodiments, the controller 210 can be disposed on, located inside, coupled to, and/or otherwise associated with one or more other components of the system 100. As shown in FIG. 3, the controller 210 can include a memory device 212, a processor 214, and a data storage component 216. Likewise, the brace 102 can include a controller 220 disposed on, coupled to, and/or otherwise associated with it. Similar to the controller 210, in alternative embodiments, the controller 220 can be disposed on, coupled to, woven into, and/or otherwise associated with the brace 102. As shown in FIG. 3, the controller 220 can include a memory device 222, a processor 224, and a data storage component 226. Upon use of the brace system 100, the processor 224 in the controller 220 can be operable to receive signals indicating brace parameters from one or more operating brace sensors 118. The processor 224, in turn, processes the signals, which can occur, for example, based on instructions in the memory device 222, and the processor 224 can then determine patient brace data from the processed data. Patient brace data may include but is not limited to knee angle, knee location, patient posture, muscle tone, swing time, or toe touch force data, brace stiffness, brace temperature, brace pressure, degree or motion, or brace energy. The processor 224 can determine the patient brace data solely based on the signals received, and/or other information can be provided that can be used in tandem with the signals to derive the patient brace data. Similarly, the processor 214 in the controller 210 can be operable to receive signals indicating crutch parameters from one or more operating crutch sensors 116. The processor 214, in turn, processes the signals, which can occur, for example, based on instructions in the memory device 212, and the processor 214 can then determine patient gait data, or other data as would be understood by a person skilled in the art depending, at least in part, on the action(s) being monitored, from the processed data. Patient gait data may include but is not limited to patient gait phase event data, patient gait timing data, patient joint movement data, ground reaction force, patient gait characteristics, patient gait phase event data, patient gait timing data, patient joint movement, ground reaction force, fall prediction, fall detection, foot usage data, crutch energy, or any other data indicative of patient mobility. Patient gait data and patient brace data may include information, electrons, energy, and/or signals.

The controllers 210, 220 can be configured to be operable to receive data anytime during the day and/or the night. The controllers 210, 220 can be configured to be operable to receive data continuously and/or intermittently, for example at fixed time intervals. For example, the controllers 210, 220 can be configured to be operable to receive data on a daily, nightly, hourly, and/or weekly basis. In some embodiments, different types of data may be received at different times by the controllers 210, 220. In some embodiments, both the controllers 210, 220 can be configured to be operable to receive data at the same time. In other embodiments, the controllers 210, 220 can be configured to be operable to receive data at different times.

Referring again to FIG. 1, the data hub 120 can be located on the crutch 104 and can communicate with the controllers 210, 220, for example via Bluetooth® wireless technology, or by other wired or wireless technologies. In some embodiments, the data hub 120 may be located on the brace 102 instead of, and/or in addition to, being located on the crutch 104. In some embodiments, the data hub 120 serves as the temporal IoT (internet of things) edge for data collection, processing, and computation before periodically updating the next IoT edge 160, which, as shown, can be a smart device 166 like a smart phone, a computer system, the cloud, and/or any other mobile or computational device capable of storing, receiving, and/or transmitting data. Still further, the initial data hub 120 can be part of a smart device such that the controllers 210, 220 communicate directly with the smart device included as part of the brace system 100. The next IoT edge 160 can serve both as a data storage hub and/or a portal to upload relevant data into one or remote databases, such remote databases being physical storage locations (e.g., a hard drive at a data center) or virtual (e.g., the cloud). The next IoT edge 160 can be used to access the rehabilitation application 230 used by the patient. The data hub 120 and/or the temporal IoT edge can filter and/or process data so that only relevant data gets uploaded to the next IoT edge 160 and/or is accessible to the rehabilitation application 230 used by the patient. The data hub 120 and/or the temporal IoT edge(s) can also mitigate data collection and storage when the next IoT edge 160 is not available. In some embodiments, data exchange between the next IoT edge 160 and the data hub 120 and/or the temporal IoT edge can happen at charging. The controllers 210, 220 can be operable to communicate any processed data and/or unprocessed data, including data determined by passive and/or active tracking to the data hub 120 and/or to the rehabilitation application 230 used by the patient. In some embodiments, the patient may access their rehabilitation protocol through the rehabilitation application 230 on an external device in addition to, or instead of, the next IoT edge 160. A person skilled in the art will appreciate many other data communication set-ups that can be used without departing from the spirit of the present disclosure, with various sensors, controllers, data hubs, and IoT edges, among other components of a data transmission system, being able to be moved to different locations, added, removed, etc.

Referring to FIGS. 1 and 3, the brace 102 and the crutch can communicate with each other directly and/or through the data hub 120. The controller 220 associated with the brace 102 can be operable to communicate the patient brace data and/or brace parameters to the crutch 104, and/or components thereof, and the controller 210 associated with the crutch 104 can be operable to communicate the patient gait data and/or crutch parameters to the brace 102, and/or components thereof. In some embodiments, the controllers 210, 220 may be operable to communicate unprocessed data between the brace 102 and crutch 104. The communication of the unprocessed data, patient gait data, patient brace data, brace parameters, and/or crutch parameters (hereinafter also referred to collectively as data) between the brace 102 and crutch 104 can be dependent, at least in part, on a positional relationship and/or proximity between the brace 102 and the crutch 104. The proximity between the brace 102 and the crutch 104 during different stages of patient ambulation is illustrated by action 130, action 140, and action 150 in FIG. 1. As shown, the brace 102 and the crutch 104 are located in close proximity to each other at certain times during the patient gait cycle (e.g., action 140), and are located further away from each during other times (e.g., actions 130, 150). Actions 130 and 150 illustrate brace signals 234 generated by the brace 102, and action 140 illustrates both the brace signals 234 generated by the brace 102 and crutch signals 236 generated by the crutch 104. Communication between the brace 102 and the crutch 104 can occur via the brace signals 234 and the crutch signals 236 when the brace 102 and the crutch 104 are in close proximity to each other and the distance between them is approximately in the range of about 1 inch to about 18 inches, including any distance or range comprised therein. The foregoing notwithstanding, ranges larger than 18 inches may be possible, including significantly larger than 18 inches, without departing from the spirit of the present disclosure. In one non-limiting embodiment, the distance between the brace 102 and crutch 104 is approximately in the range of about 6 inches to about 12 inches, including any distance or range comprised therein. In some embodiments, communication between the brace 102 and the crutch 104 can occur as long as the brace 102 and the crutch 104 are paired and the two processors 214, 224 are within a communicating distance permitted by technology, such as Bluetooth® wireless technology. The signals 234, 236 can be broadcast in any manner known to those skilled in the art, including being selectively activated or constantly sending the signals 234, 236. For example, the signals 234, 236 can be broadcast on a constant basis (i.e., always on), on a consistent basis (i.e., on at regular time intervals and/or in response to one or more designated triggers), on an intermittent basis (i.e., at time intervals that are not necessarily consistent and/or in response to one or more designated triggers), on an automated basis (i.e., in response to designated times and/or other triggers), and/or on a manual basis (i.e., in response to a user commanding the broadcast), among other ways by which signals can be broadcast and/or otherwise communicated.

The processor 214, 224 can be capable of processing instructions for execution within the brace system 100. The processor 214, 224 can be a single-threaded processor, a multi-threaded processor, or similar device. The processor 214, 224 can be capable of processing instructions stored in the memory 212, 222 or on the data storage 216, 226. The memory 212, 222 can store information within the system 100. In some implementations, the memory 212, 222 can be a computer-readable medium. The memory 212, 222 can, for example, be a volatile memory unit or a non-volatile memory unit. In some implementations, the memory 212, 222 can store information related to the patient, including but not limited to information specific to the patient on an individual level, as well as information related to demographics of the patient more generally, such as information that relates to how patients having similar characteristics and/or profiles have responded to treatments of a similar nature.

The data storage 216, 226 can be capable of providing mass storage for the system 100. In some implementations, the data storage 216, 226 can be a non-transitory computer-readable medium. The data storage 216, 226 can include, for example, a hard disk device, an optical disk device, a solid-date drive, a flash drive, magnetic tape, and/or some other large capacity storage device. The data storage 216, 226 may alternatively be a cloud storage device, e.g., a logical storage device including multiple physical storage devices distributed on a network and accessed using a network. In some implementations, the information stored on the memory 212, 222 can also or instead be stored on the data storage 216, 226.

Although examples of the brace system 100 have been described in this disclosure, implementations of the subject matter and the functional operations described herein above can be implemented in other types of digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible program carrier, for example a computer-readable medium, for execution by, or to control the operation of, a processing system. The computer readable medium can be a machine-readable storage device, a machine-readable storage substrate, a memory device, a composition of matter effecting a machine readable propagated signal, or a combination of one or more of them.

The term “computer” or “computer system” may encompass all apparatus, devices, and machines for processing data, including, by way of non-limiting examples, a programmable processor, a computer, or multiple processors or computers. A processing system can include, in addition to hardware, code that creates an execution environment for the computer program in question, e.g., code that constitutes processor firmware, a protocol stack, a database management system, an operating system, or a combination of one or more of them.

While a person skilled in the art will appreciate a variety of different types of sensors and/or methods of data gathering and transferring that can be used in conjunction with the system 100, in some embodiments the brace 102 and/or the crutch 104 can communicate data by Hall Effect magnets. In some such embodiments, the distance between the brace 102 and the crutch 104 for which communication can be achieved can be approximately in the range of about 1 inch to about 6 inches, including any distance or range comprised therein. The foregoing notwithstanding, ranges larger than 6 inches may be possible without departing from the spirit of the present disclosure. Data communicated between the components of the brace system 100 such as the brace 102 and the crutch 104 may be via signals (e.g., brace signals 234, crutch signals 236), electrons, and/or any other data transmission techniques known to those skilled in the art. Data may be communicated between the brace 102 and the crutch 104 as video transmissions, audio transmissions, and/or any other types of transmission techniques known to those skilled in the art.

The crutch sensors 116 can be designed to recognize the brace 102 by the detection of physical markers on the brace 102. Similarly, the brace sensors 118 can be designed to recognize the crutch 104 by the detection of physical markers on the crutch 104. Thus, the brace system 100 can be used for passive and/or active tracking of the patient's joint movement (e.g., joint angles) or gait based on visual detection of physical markers. Such passive or active tracking can be done by using inverse kinematics or inverse dynamics, among other techniques known to those skilled in the art. By detecting inertial values from the crutch 104 and based on crutch 104 dimensions, patient arm location can be estimated. Further, the knee location can be derived based on and/or from the patient arm location. Still further, a detail record of gait can be derived based on and/or from the inertial sensing from the brace 102 and/or from detailed tracking of the brace 102. The controllers 210, 220 can be operable to detect the physical markers on the brace 102 and crutch 104 automatically and/or manually. Automatic detection can be used for real-time assessment of a patient's joint movement and/or gait, among other patient parameters. Alternatively, or additionally, either or both of the brace 102 and the crutch 104, including but not limited to their respective sensors 118, 116, can utilize other devices or techniques known to those skilled in the art for purposes of detecting the type, design, or other features of the counterpart brace or crutch with which it is being used. By way of non-limiting example, either or both can include RFID tags that allow information related to the same to be detected by the other object without a user or other person identify the respective brace(s) and/or crutch(es), thereby allowing a brace(s) to recognize the type of crutch(es) with which it is being used and/or the crutch(es) to recognize the type of brace(s) with which it is being used without outside intervention.

In some embodiments, energy may be communicated between the brace 102 and crutch 104. Energy transfer between the brace 102 and the crutch 104 may be operable to power and/or charge components associated with the brace 102 and/or crutch 104, such as the brace sensors 118, crutch sensors 116, actuators 122, and/or electrodes 124. In some embodiments, the brace 102 or the crutch 104 may have a battery and/or a different, non-battery energy source, and/or may be operable to be powered and/or charged by an external power source. In such embodiments, the range to provide power and/or charging can be similar to the range at which data communication can be performed.

FIG. 3 is a flowchart 301 that shows one embodiment of a possible relationship between the different components of the brace system 100 provided for by the present disclosure. Data collected by the crutch sensors 116 and/or the brace sensors 118 can be communicated to the data hub 120 and then to the rehabilitation application 230. As noted above, in other embodiments the data hub 120 and the app 230 can be one in the same, either on a separate smart device or incorporated onto one of the components of the system (e.g., the crutch 104). The rehabilitation application 230 can include one or more algorithms (e.g., data processing, machine learning algorithms, federate learning), operable to process the data communicated from the crutch 104 and/or the brace 102 and to produce response outputs 240 that are communicated to the brace 102, the crutch 104, the patient, and/or the caregiver. The algorithm(s) can also be operable to use information provided by the caregivers 258 in real-time and/or over a period of time as an input. Additionally, or alternatively, the response outputs 240 can be communicated to a database 252 that assimilates response outputs 240 from a general population of users of the brace system 100. The database 252 can include depersonalized data 250 from multiple patients, which can be used to better inform the effectiveness of treatment plans across the population, based, for example, on the type of treatments, the response to the same, the demographics of the population, and/or other information a person skilled in the art will appreciate can impact the effectiveness of a rehabilitation protocol for a patient. Depersonalized data 250 from the database 252 can be directly accessed by the rehabilitation application 230. An AI system accumulating, processing, and/or analyzing such population based response output 240 may transfer the information to the rehabilitation application 230 independent of any caregiver input.

Algorithms in the rehabilitation application 230 can assess the patient's rehabilitation progress and identify potential needs or determine response outputs 240 for modifying the patient rehabilitation protocol. The response output 240 produced by the rehabilitation application 230 can be an active response and/or a passive response. The passive response can include feedback 244 and/or a summary or analysis 246. The active response can include direct communication with the brace 102 and/or crutch 104. The response outputs 240 produced by the rehabilitation application 230 can include, by way of non-limiting examples: response signals 242 provided to the brace 102 and/or crutch 104 that can lead to adjustments of the brace 102 and/or crutch 104, as described in greater detail below; notification feedback 244 provided by warnings to the patient 244 (e.g., audible feedback, visual feedback, such as by way of a notification delivered on the app 230 to a user's smart device, tactile feedback, such as by way of one of the actuators 122 on the brace 102, etc.); and/or a summary or analysis 246 including any changes and/or suggestions made to the rehabilitation protocol, and/or an indicator communicated to medical records 248. For example, if the patient's mobility is more limited than it should be, the rehabilitation application 230 may determine that the patient could benefit from a more aggressive rehabilitation protocol and provide the patient a recommendation to increase their activity level by providing a summary or analysis 246 indicating as such. A person skilled in the art, in view of the present disclosures, will understand other possible outputs that can be produced by the rehab application 230 in response to data received from the data hub 120 and/or clinical input 258.

Algorithms operable by the rehabilitation protocol 230 can also be used to alter one or more brace properties, including but not limited to stiffness, the degree of motion, and the temperature of the brace 102. The one or more brace properties that can be altered can mirror one or more of the properties monitored by the sensors 118, or they can be one or more different properties of the brace. The response outputs 240 produced by the rehabilitation application 230 and communicated to the brace 102 include response signals 242 to mechanically, electrically, and/or thermally stimulate or otherwise impact or adjust the brace 102. The response signals 242 may induce a haptic response from the brace 102. The actuators 122 and/or electrodes 124 associated with the brace 102 can be configured to be operable to adjust, change, and/or redefine the brace properties based on the response signals 242. For example, active heating or cooling of the brace 102 to aid in patient rehabilitation can be turned on or off automatically or manually through the rehabilitation application 230. The location and intensity of a stimulus provided via the output 240 altering the characteristics of the brace 102 can be adjusted or altered based, at least in part, on clinical input 258, population data communicated to the database 252, and/or data collected from the crutch sensors 116 and/or brace sensors 118 and communicated through the data hub 120. The brace 102 may be stimulated for attentional focus, which can, for example, gather awareness for improving rehabilitation performance and/or reducing re-injury.

Likewise, the response outputs 240 produced by the rehabilitation application 230 and communicated to the crutch 104 can include response signals 242 to mechanically, electrically, and/or thermally stimulate or otherwise impact or adjust the crutch 104. Additionally, or alternatively, the response signals 242 may induce a haptic response from the crutch 104. The location and intensity of a stimulus provided via the output 240 altering the characteristics of the crutch 104 can be adjusted or altered based, at least in part, on clinical input 258, population data communicated to the database 252, and/or data collected from the crutch sensors 116 and/or brace sensors 118 and communicated through the data hub 120.

In some embodiments, the brace 102 of the brace system 100 can have a manual “override” button so that the patient can ensure that any angle variation of his or her brace 102 is either held in extension or at least biased towards extension when, for example, he or she is walking in a crowd or on uneven terrain. The override button can be used to override any rehabilitation protocol, or subset of such protocol. The brace 102 can be designed to auto-override and go to locked full extension if the brace sensors 118 and/or the crutch sensors 116 determine that the patient is stumbling or falling.

Referring to FIG. 3, the output response 240 may be a notification feedback 244 delivered to the patient as a warning. Algorithms operable by the rehabilitation application 230 can detect potentially hazardous patient postures based, at least in part, on clinical input 258, population data communicated to the database 252, and/or data collected from the crutch sensors 116 or brace sensors 118 and communicated through the data hub 120, and then issue alerts to the patient. For example, a warning may be issued predicting or detecting that a fall may or has occurred based on patient brace data and/or patient crutch data about patient posture and/or gait data, among other information that can be detected by the sensors 116, 118. Additionally, or alternatively, a warning may be issued when overloaded by placing too much body weight on the extremity being treated is detected, and/or when the patient is trying to extend the knee beyond a safe range for a particular phase of recovery.

The response output 240 can be communicated as a summary or analysis 246 to the patient. The summary or analysis 246 can be determined by the algorithms operable by the rehabilitation application 230 based, at least in part, on data collected from the crutch sensors 116 and/or brace sensors 118 and communicated through the data hub 120 over a period of time. The period of time can be approximately in the range of about 1 day to about the number of days of usage of the brace system 100 by the patient (e.g., 120 days, 180 days, 365 days or greater), including any number of days or range comprised therein. The summary or analysis 246 can summarize, tabulate, and/or predict patient progress. The patient and/or the caregiver can use the summary or analysis 246 determined by the rehabilitation application 230 to refine and/or readdress a part of the patient's rehabilitation protocol. Additionally, or alternatively, the response output 240 can be communicated as a daily and/or weekly summary of patient activity as mandated by the patient protocol and/or as completed by the patient.

If the output response 240 is an indicator communicated to medical records 248, it can be used in a variety of ways. In some instances, it can be used to treat the individual patient. That is, the output response 240 can be communicated to a clinician, for example at a visit 256, and then can provide input 258 to make adjustments to the rehabilitation protocol provided for by the app 230. Alternatively, the clinic visit 256 does not have to be an actual physical visit, but rather, it can be a virtual visit, or just a virtual input more generally without a live interaction between the patient and the clinician. The virtual input also possible at action 256 can be a clinician receiving the output 248, providing relevant input 258 in view of the same, and making an adjustment to the rehabilitation protocol that is communicated to the app 230, all without direct patient-to-clinician interaction. The input provided by the clinician can provide for ongoing assessments and/or recommendations without requiring clinical visits.

Another way the output response 240 can be used is to provide clinical data more generally, helping to populate a medical database(s) that can inform treatments across a population. As shown and described previously, the output response in the form of information communicated to medical records 248 can be converted to depersonalized data 250 and entered into the database 252. Accordingly, the depersonalized data 250 in the database 252 can be used to evaluate and provide outcome prediction services 254 256. As shown, the caregiver may use both the response data 240 about the individual patient that is communicated to the medical records 248, as well as information assessed from the outcome prediction service 254 about the patient population more generally, to provide clinical input 258 during an in-person visit and a virtual visit. Additionally, or alternatively, the caregiver can provide the clinical input 258 to the rehabilitation application 230 outside of a visit 256, allowing for changes to be made to a rehabilitation protocol at any time, without requiring a clinic visit. Such clinical input 258 can be used by the algorithms in the rehabilitation application 230 during ongoing assessments.

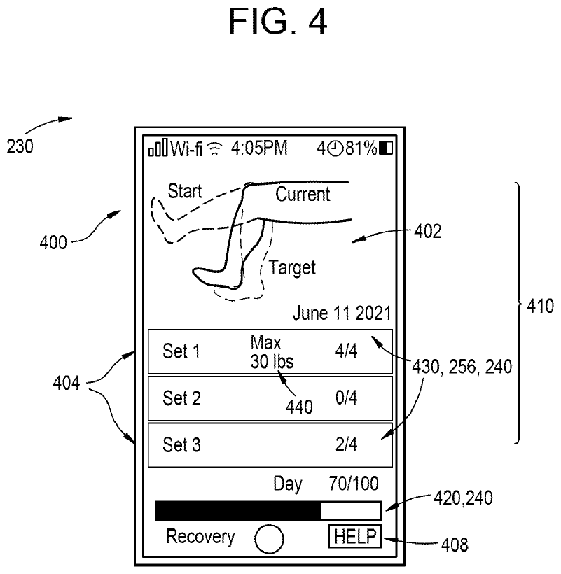

As described earlier, the patient can use the next IoT edge 160 to access the rehabilitation application 230. FIG. 4 illustrates one embodiment of a user interface 400 associated with the rehabilitation application 230. The rehabilitation application 230 used by the patient can include the user interface 400, which can be configured to illustrate a rehabilitation protocol 410 for the patient, a record of the patient's activity 430, an assessment of the patient's ability 440, and/or one or more of the response outputs 240 determined by an algorithm. As shown in FIG. 4, the patient's ability 440 can be determined based, at least in part, on the rehabilitation protocol 410. In the illustration of FIG. 4, the rehabilitation protocol 410 shows a diagrammatic representation 402 and an exercise regimen or rehabilitation plan 404 for the patient. For example, the patient may engage in three (3) sets of the weight bearing exercises shown in the diagrammatic representation 402, and each set may include four attempts as shown in the rehabilitation plan 404. The patient's weight bearing ability 440 has been assessed to be at a maximum of 30 lbs. The one or more response outputs 240 can include a progress bar 420 and a summary or analysis of patient activity 256. The summary or analysis of patient activity 256 in the illustrated embodiments shows that the patient performed all four attempts of set one, no attempts of set two, and two attempts of set three. The progress bar 420 illustrates that the patient is currently on the seventieth (70^{th}) day of a one-hundred (100) day rehabilitation protocol 410. The patient can access more information about the rehabilitation protocol 410 by executing the help button 408.

The rehabilitation protocol 410 can be optimized in a manner as described above with respect to FIG. 3, meaning it can be adjusted based, at least in part, on clinical input 258, population data communicated to the database 252, and/or data communicated from the data hub 120. For example, the rehabilitation protocol 410 can depend on the post-surgery time period that a patient has been engaging in rehabilitation, and thus, may include simple tasks at an early stage and more challenging tasks at a later stage. This can be important in determining a rehabilitation protocol 410 that requires the patient to enact full range of knee motion at an early recovery stage, and perform weighted tasks during the latter stages for strength training and evaluation. The rehabilitation application 230 can record and/or display patient gait data and patient brace data on any time scale, including instantaneously, daily, and/or weekly bases.

The algorithms operable in the rehabilitation application 230 can be automatically or manually adjusted to be more or less aggressive depending, at least in part, on the patient progress. For example, the brace 102 may be locked for one patient for 6 days, and 9 days for a different patient. Thus, the algorithms in the rehabilitation application 230 may be personalized based on patient recovery, patient motivation, and/or patient compliance.

By way of non-limiting example, algorithms that can be implemented in view of the present disclosure using systems and methods discussed are listed below. Further, a person skilled in the art, in view of the disclosures, will understand other embodiments of the examples listed below. A person skilled in the art, in view of the present disclosures, will understand how to implement different embodiments of the systems and algorithms described.

### EXAMPLE 1: LOAD BALANCE ALGORITHM

In one exemplary embodiment, a load balance algorithm operable by the rehabilitation protocol 230 can be used to alter the stiffness or freedom of motion of the brace 102. With reference to FIGS. 1 and 3, data such as the force and orientation of the crutch tip 108 measured by the crutch sensors 116 can be communicated to the rehabilitation application 230 through the data hub 120. A load balance algorithm in the rehabilitation application 230 can be operable to calculate the potential load on the patient's knee and determine an appropriate stiffness or freedom of motion of the brace 102 as a response output 240 and communicate the same to the controller 220 by response signals 242. The controller 220 can be operable to activate the actuators 122 and/or electrodes 124, among other features of a brace capable of making adjustments to the brace 102, to adjust the stiffness or freedom of motion of the brace 102.

### EXAMPLE 2: MUSCLE STIMULATION

In another exemplary embodiment, a patient's joint movement information and force or EMG data from a toe touch can be used for active and/or passive muscle stimulation in the region protected by the brace 102. Again with reference to FIGS. 1 and 3, the response output 240 determined by the rehabilitation application 230 based on the data communicated to it can be automatically transmitted as response signals 242 to the controller 220 in the brace 102. The controller 220 can then operable to activate the actuators 122 and/or electrodes 124 to timely stimulate the correct muscles in the patient. Alternatively, or additionally, the patient and/or caregiver can assess how certain muscles are stimulated during the recovery process based on the summary or analysis 246 provided by the rehabilitation application 230 over a certain period of time. In some embodiments, the patient and/or the caregiver can use the response output 240 determined by the rehabilitation application 230 and communicated as an audible output 244 or as an analysis 246 to manually affect the actuators 122 and/or electrodes 124, among other features of a brace capable of making adjustments to the brace 102, to timely stimulate the correct muscles in the patient. Algorithms operable by the rehabilitation protocol 230 can include a sensory guide to enable the determination of the correct muscle for stimulation.

### EXAMPLE 3: ACL RECONSTRUCTION

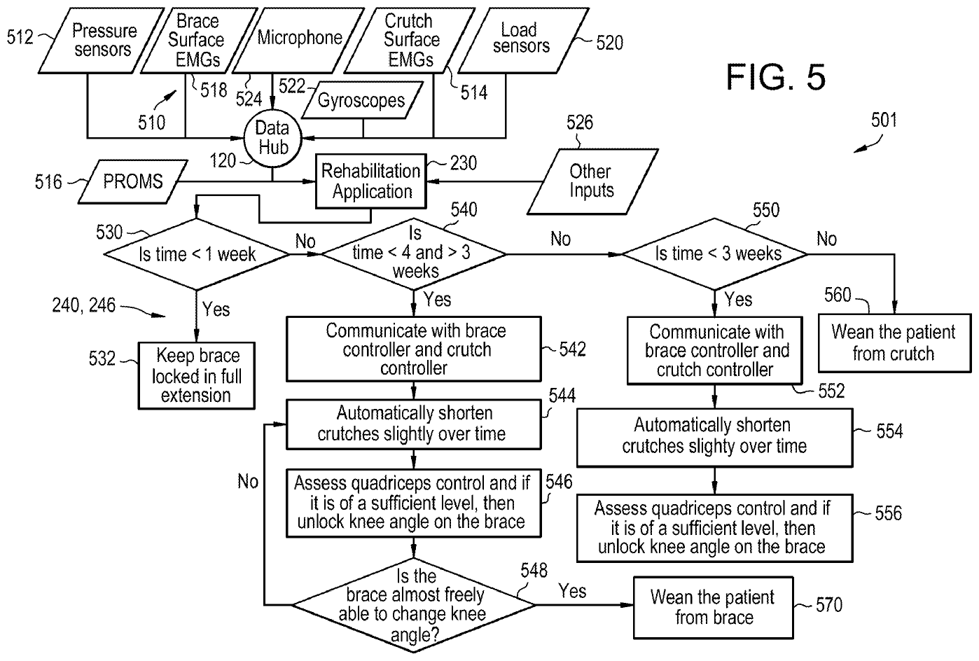

In still another exemplary embodiment, the brace system 100 described above can be used for determining and/or adjusting post-operative treatment following ACL reconstruction. FIG. 5 shows a flowchart 501 that illustrates an algorithm that can be used for determining and/or adjusting post-operative treatment following ACL reconstruction. Reference is made herein to features of the system 100 as provided for in FIGS. 1 and 3, although such references are non-limiting. The flowchart 501 identifies non-limiting examples of different inputs 510 that can be provided to the rehabilitation application 230. The inputs can include data provided by pressure sensors 512 that can be disposed, for example, along a proximal or upper portion of a rim of the brace 102, surface EMGs 514 that can be disposed on the crutch 104, patient recorded outcome measures (PROMs) 516 associated with any part of the system 100 or the patient more generally, surface EMGs 518 on the brace 102, as well as load sensors 520, gyroscopes 522, and/or microphones 524 associated with any part of the system 100 or the patient more generally. By way of example, the pressure sensors 512, surface EMG 518, and PROMs 516 can be configured to collect data about muscle activation and firing in the patient's quadriceps.

The controller 220 in the brace 104 can be operable to determine the evenness of quadriceps firing based, at least in part, on the collected data. The PROMs 516 can include, by way of non-limiting example, patient feedback regarding pain, leg fatigue, ease of getting out of a chair, and/or ability to ambulate, such information being able to be entered into the rehabilitation application 230 by the user. The surface EMG 514 on the crutch 104 can be configured to assess, among other information, patient gait mechanics based, for example, on the timing of quadriceps muscle firing during ambulation and/or based on the activity of the patient's soleus muscle. The pressure sensors 512 can be configured to determine whether the patient leans forward at the beginning of the stance phase to lock his or her knee. One or more load sensors 520 can be configured to determine the patient's weight bearing percentage and/or to gauge whether the patient is using one crutch or two crutches 104, 106, among other determinations. The microphone 524 can be configured to identify the patient stumbling or falling, among other determinations, and the gyroscope 522 can be configured to determine any abrupt change in the patient's position, among other determinations. The controllers 210, 220 can be operable to receive the crutch signals 236 from the operating crutch sensors 116 and/or brace signals 234 from the operating brace sensors 118, process the crutch signals 236 and brace signals 234, and communicate the crutch signals 236 and the brace signals 234 to the rehabilitation application 230, via the data hub 120 in at least some instances. The rehabilitation application 230 can also be configured to use clinical input 258, and other inputs 526, such as the elapsed time since surgery and/or population-based information as determined from a global database, in determining and/or adjusting the post-operative treatment following ACL reconstruction.

The rehabilitation application 230 can be operable to receive the inputs 510, process the inputs 510, and determine a response output 240. FIG. 5 provides a non-limiting example of such an output 240, which also provides for a summary or analysis 246 that can be communicated to the user. Other outputs, such as those described above with respect to FIG. 3 (e.g., response signals 242, feedback 244), are also possible. In step 530, the rehabilitation application 230 can determine if the elapsed time since surgery 526 is less than one week. If the determination is in the affirmative, the response output 240 generated by the rehabilitation application 230 can include a summary or analysis 246 communicated to the patient in step 532. In the illustrated embodiment, the analysis 246 indicated by the rehabilitation application 230 is that the patient keep the brace 102 locked in full extension.

If the elapsed time since surgery 526 is more than one week, the rehabilitation application 230 can determine in step 540 if the elapsed time is greater than three weeks and less than four weeks. As shown, if the determination is in the affirmative, the response output 240 generated by the rehabilitation application 230 can include response signals (e.g., the response signals 242 from FIG. 3) communicated to the controller(s) 210, 220 in step 542. For example, as shown in step 544, the controller 210 can be operable to automatically shorten the crutch 104 slightly over time. This change in height of the crutch 104 encourages gradual progression of weight bearing. For example, a short crutch height can comprise a length when the crutch 104 is not supporting the patient's body weight. The short crutch height may be slightly less than the distance from the ground to the patient's axilla (armpit). The process of changing the height of the crutch 104 typically takes place over the course of multiple weeks, so the change in height can be approximately in the range of about 0.1 mm to about 25 mm per day, and in some instances it can be about 1 mm per day or less than 1 mm per day. The foregoing notwithstanding, change in height of the crutch 104 at a rate faster than 25 mm per day or slower than 0.1 mm per day may be possible without departing from the spirit of the present disclosure. Additionally, or alternatively, the change of height of the crutch 104 can be measured per period of time such as per 6 hour period, per 12 hour period, etc.

By way of further non-limiting example, higher quadriceps control can indicate that the brace 102 should be configured to permit the patient to make an easier change to his or her knee angle. Thus, in step 546, the controller 220 can be operable to automatically assess quadriceps control and if it is of a sufficient level (e.g., of a clinically safe and/or determined strength, such as can be determined by a physician, physical therapist, and/or other rehabilitation specialist), then the controller 220 is operable to unlock the knee angle on the brace 102 by communicating with the actuators 122. The sufficient level can be determined by visits to the caregiver or may be based on the patient's progress. The ease with which the brace 102 permits knee angle changes can be variable and can vary in flexion, extension, or both. In step 548, the controller 220 can be operable to determine if the brace 102 is almost freely able to change knee angle. For example, the knee angle can be free to change within specific limits as determined by the controller 220 instead of being locked to one specific angle, such as full extension. If the controller 220 determines that the brace 102 is not freely able to change knee angle, the controller 220 can communicate with the controller 210 to execute step 542 again. If the controller 220 determines that the brace 102 is freely able to change knee angle, the controller 220 can communicate with the rehabilitation protocol 230 to execute step 570. In step 570, the rehabilitation protocol 230 can include the response output 240 that contains a summary or analysis 246 communicated to the patient. For example, as shown, the patient can be instructed to wean him or herself of the brace 102, including to stop using the brace 102.

In step 540, if the rehabilitation application 230 determines that the elapsed time since surgery 526 is not greater than three weeks and less than four weeks (e.g., it is two weeks or five weeks), the rehabilitation application 230 can determine in step 550 if the elapsed time is less than three weeks. If the determination is in the affirmative (e.g., it is two weeks), the response output 240 generated by the rehabilitation application 230 can include response signals 242 that can be communicated to the controllers 210, 220 in step 552. In step 554, the controller 210 can be operable to automatically shorten the crutch slightly over time. Further, in step 556, the controller 220 can be operable to automatically assess quadriceps control and, if it is of a sufficient level, then the controller 220 can be operable to unlock the knee angle on the brace 102 by communicating with the actuators 122 and/or electrodes 124, among other features that may be part of the brace 102. In step 550, if the rehabilitation application 230 determines that the elapsed time since surgery 526 is not less than three weeks (e.g., it is five weeks), the rehabilitation application 230 can execute step 560. In step 560, the rehabilitation protocol 230 can include the response output 240 that contains a summary or analysis 246 communicated to the patient to stop using the crutch 104.

### EXAMPLE 4: DATA COLLECTION AND DECISION FLOW

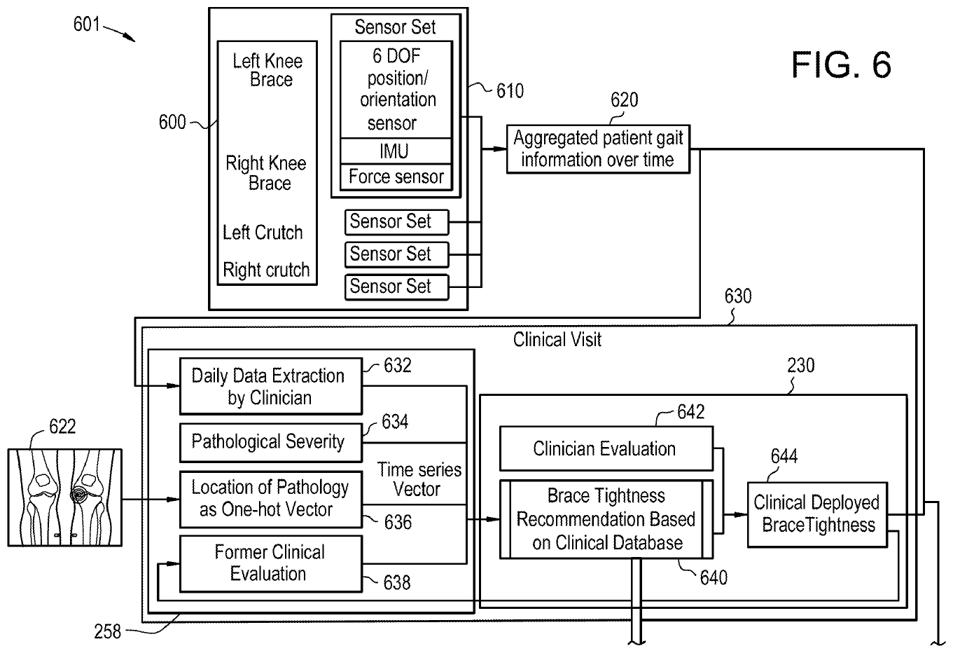

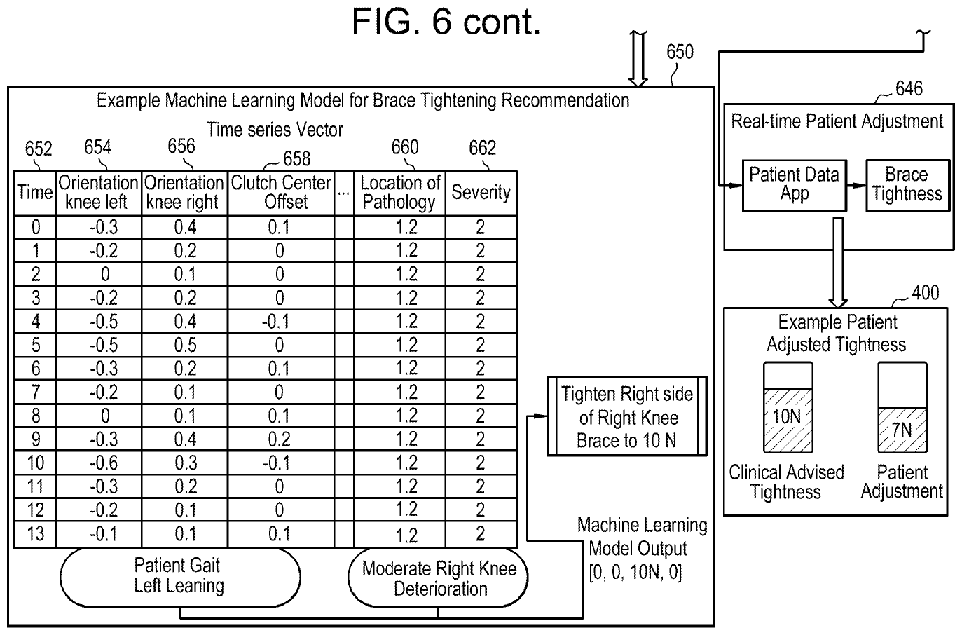

In one exemplary embodiment, a brace system 600 can be used for collecting data relevant for treating knee impairment. The brace system 100 can also be used in a similar manner. FIG. 6 shows a flowchart 601 that illustrates one non-limiting embodiment of a relationship and interaction between data collected by sensors, caregiver assessments of the patient being evaluated and/or of other patients made by the same caregiver or a different caregiver, information stored in clinical databases, and algorithms operable in a rehabilitation application, such as the rehabilitation app 230, in determining changes to a patient rehabilitation protocol.

The brace system 600 provided for in FIG. 6 comprises two braces and two crutches. The brace sensors on the left and right brace, and crutch sensors on the left and right crutches can be operable to measure data relevant to determine brace stiffness 610. The measured data can be aggregated over time, for example in a data hub 120 and/or communicated to the rehabilitation application 230. The aggregated data 620 can be assessed by the caregiver, for example during a clinic visit 630 or at other times as provided for herein, and used along with patient assessments 622, such as X-ray, magnetic resonance imaging (MRI), etc. The clinic visit 630 may be in-person, conducted via video- or tele-medicine, or using other ways by which a clinician and user can communicate. Certain steps of the clinic visit 630 can include extraction of the daily data aggregated by the sensors 632 (or across other designated periods of time, such as hourly, weekly, etc.), assessment of pathological severity of the patient 634, and/or the identification of pathology as one-hot vector 636 can be performed by the caregiver using techniques known to those skilled in the art. The information determined in steps 632, 634, and 636 can be used as a time series vector(s), along with any prior clinical assessment(s) or evaluation(s) 638, as clinical inputs 258 to the rehabilitation application 230. As described herein, the prior clinical assessment(s) can include those of the patient being treated and/or can be aggregated from a broader population of patient data that is applicable to the patient being treated, such as because of the patient's injury, demographics, and/or one or more other points of commonality between the patient and others in the broader population.

Furthermore, the rehabilitation application 230 can use information in one or more clinical databases and one or more machine learning algorithms to determine performance characteristics for the brace(s), such as providing a brace stiffness and/or a brace tightness recommendation 640. This brace tightness recommendation 640 generated by the rehabilitation application 230 can be used to determine the brace tightness clinically deployed action 644 by the caregiver after clinical evaluation 642. The brace tightness clinically deployed action 644 can be used to make real-time adjustment actions 646 to the patient brace, for example through the user interface 400 of the rehabilitation application 230. In some embodiments, information in clinical databases and machine learning algorithms can be used to determine and/or adjust various aspects of the brace system 600, such as a height of the crutch, manually and/or automatically. In some embodiments, the determination of height of the crutch and/or the brace stiffness, and/or a brace tightness recommendation 640 may be done simultaneously or consecutively.

In this example, a machine learning algorithm can determine if the patient has been leaning on one foot or using both feet by comparing values of the brace sensors to the crutch sensors in real-time and over a period of time, as detailed by action 650. The action 650 can include, by way of non-limiting example, collection of data, including orientation of left knee 654 and orientation of right knee 656 at different time points 652, to calculate a corresponding clutch center offset 658, which can be used to determine if the patient gait is left leaning or right leaning. A skilled caregiver and/or the rehabilitation application 230 can use the recorded data, clinical information on the location of pathology 660, and/or clinical information on the severity of pathology 662 to generate a brace tightness recommendation 640. The information can be fit into a statistical model (e.g., linear fitting) to determine a tightening location and/or value can be suggested to the patient or caregiver in action 644.

For example, if the patient gait is left leaning with a moderate right knee deterioration, as shown in FIG. 6, the caregiver and/or the rehabilitation application 230 can generate the brace tightness recommendation 640 to tighten the right side of right knee brace up to 10 N. The brace tightness recommendation 640 can be clinically deployed 644 to the patient by the caregiver and/or directly by the rehabilitation application 230. The brace tightness recommendation 640 can be provided in real-time and/or stored in a patient database for later use. The patient can then access the brace tightness recommendation 640 to adjust the brace tightness in action 646. The patient may employ the force suggested (e.g., 10 N) by the brace tightness recommendation 640 or a force lower (e.g., 7 N) than the suggested force as shown in the user interface 400.

The examples discussed above are not limiting and a person skilled in the art, in view of the disclosures, will understand other ways by which the brace system 100, 600 can be operated, and other means by which data may be collected, communicated, and/or assessed by algorithms in the rehabilitation application and/or by a caregiver. Furthermore, a person skilled in the art, in view of the disclosures, will understand other ways by which an algorithm can be operated to actively or passively control one or more components the brace system 100, 600.

Some non-limiting examples of the above-described embodiments can include the following:

1. A patient brace system, comprising:
a crutch;
at least one crutch sensor configured to measure one or more parameters associated with the crutch and generate crutch signals in view of the same;
a controller operable to receive the generated crutch signals from the at least one crutch sensor, and further operable to process the generated crutch signals to produce patient gait data and communicate at least one of the one or more parameters measured by the at least one crutch sensor or the patient gait data to a data hub;
a patient brace; and
a rehabilitation application in communication with the data hub and configured to receive a clinical input, the rehabilitation application being configured to process the clinical input and the patient gait data to determine a response output and communicate the response output to at least one of a caregiver, a user of the patient brace, or to the patient brace.
   2. The patient brace system of example 1, further comprising:
a second controller operable to receive at least one of the one or more parameters measured by the at least one crutch sensor or the patient gait data, and is further operable to process at least one of the one or more parameters measured by the at least one crutch sensor or the patient gait data and determine a load acting on at least one of the patient brace or patient knee.
   3. The patient brace system of example 1, wherein the system further comprises:
at least one brace sensor configured to measure one or more parameters associated with the brace and generate brace signals in view of the same; and
a second controller operable to receive the generated brace signals from the at least one brace sensor, and further operable to process the generated brace signals to produce patient brace data and communicate at least one of the one or more parameters measured by the at least one brace sensor or the patient brace data to the data hub,
wherein the rehabilitation application is configured to process the patient brace data in conjunction with determining the response output.
   4. The patient brace system of example 3,
wherein the at least one crutch sensor and the controller is located on the crutch,
wherein the at least one brace sensor and the second controller is located on the brace, and
wherein the controller and the second controller are configured to transmit the patient gait data and the patient brace data, respectively, to each other.
   5. The patient brace system of any of examples 2 to 4, wherein the patient gait data and patient brace data includes one or more of information, electrons, energy, or signals.
   6. The patient brace system of any of examples 2 to 5, wherein the at least one brace sensor is operable to determine at least one of movement or location of the patient brace in relation to the crutch.
   7. The patient brace system of any of examples 2 to 6, wherein the patient brace data comprises at least one of: knee angle, knee location, patient posture, muscle tone, swing time, or toe touch force data.
   8. The patient brace system of any of examples 2 to 7,
wherein the second controller is operable to receive the output response generated by the rehabilitation application, and is further operable to process the generated output response, and
wherein the brace responds to the output response.
   9. The patient brace system of example 8, wherein the brace includes at least one of at least one actuator or at least one electrode configured to stimulate at least one of a patient muscle, change patient brace stiffness, or change freedom of patient brace movement.
   10. The patient brace system of example 9, wherein the response output is passive and includes at least one of an audible indication, a visual indication, or a tactile indication to a user.
   11. The patient brace system of example 9, wherein the response output is active and occurs in real time.
   12. The patient brace system of any of examples 1 to 11, wherein the patient gait data comprises at least one of ground reaction force or gait timing.
   13. The patient brace system of any of examples 1 to 12, wherein the data hub is located on the crutch.
   14. The patient brace system of any of examples 1 to 13, wherein the rehabilitation application is located on an electronic device accessible to the user of the patient brace.
   15. The patient brace system of any of examples 1 to 14, wherein the crutch is a first crutch, and the patient brace system further comprises a second crutch used as a standard known datum for calibration of patient gait data.
   16. A method of adjusting a knee brace, comprising:
determining patient gait data based on one or more parameters associated with a crutch;
communicating the determined patient gait data to at least one of a controller or a data hub; and
based on the communicated patient gait data, performing at least one of the following actions:
   causing a knee brace to make an auto adjustment in response to the communicated patient gait data; or
   instructing at least one of a caregiver or a user of a knee brace to adjust the knee brace.
      17. The method of adjusting the knee brace of example 16, wherein the controller is located on a knee brace, and wherein communicating the gait data depends on a positional relationship between the knee brace and the crutch.
      18. The method of adjusting the knee brace of example 16 or 17,
wherein at least one crutch sensor is configured to measure the one or more crutch parameters associated with the patient data gait, and
wherein communicating the determined patient gait data to at least one of a controller or a data hub further comprises operating the controller to process the one or more crutch parameters to produce the patient gait data.
   19. The method of adjusting the knee brace of any of examples 16 to 18, further comprising operating a second controller to process one or more brace parameters measured by at least one brace sensor on the knee brace to produce patient brace data.
   20. The method of adjusting the knee brace of any of examples 16 to 19, wherein causing the knee brace to make the auto adjustment further comprises operating at least one of at least one brace actuator or at least one electrode based on the patient gait data.
   21. The method of adjusting the knee brace of any of examples 16 to 20, wherein causing the knee brace to make the auto adjustment further comprises using the patient gait data and the patient brace data in a load balancing algorithm.
   22. The method of adjusting the knee brace of any of examples 16 to 21, wherein causing the knee brace to make the auto adjustment further comprises operating at least one of the at least one brace actuator or the at least one electrode to change at least one of a knee brace stiffness or a knee brace freedom of motion.
   23. The method of adjusting the knee brace of any of examples 16 to 21, wherein causing the knee brace to make the auto adjustment further comprises operating at least one of the at least one brace actuator or the at least one electrode to lock the knee brace.
   24. The method of adjusting the knee brace of any of examples 20 to 23, wherein causing the knee brace to make the auto adjustment further comprises operating at least one of the at least one brace actuator or the at least one electrode to change a temperature associated with at least part of the knee brace.
   25. The method of adjusting the knee brace of any of examples 16 to 24, wherein causing the knee brace to make the auto adjustment further comprises transferring energy between the crutch and the knee brace.
   26. A method for formulating a patient rehabilitation protocol comprising:
processing patient input from a data hub;
processing clinical input from a caregiver;
assessing patient gait based on the patient input and the clinical input by using at least one of inverse kinematics or inverse dynamics; and
at least one of providing or adjusting a patient rehabilitation protocol based on the patient gait,
wherein the patient input comprises at least one of patient gait data or patient brace data.
   27. The method for determining patient rehabilitation protocol of example 26, wherein if the patient gait is limited, the patient rehabilitation protocol is determined to be aggressive with an increase in activity level.
   28. The method for determining patient rehabilitation protocol of example 26 or 27,
wherein the patient rehabilitation protocol comprises a sensory guide, and
wherein the method further comprises operating the sensory guide to determine which muscle or muscles need stimulation.
   29. The method for determining patient rehabilitation protocol of any of examples 26 to 28, wherein the patient rehabilitation protocol is accessible to a user by an application on at least one of a computer or a smart device.
   30. The method for determining patient rehabilitation protocol of any of examples 26 to 29, wherein providing or adjusting a patient rehabilitation protocol further comprises providing a user at least one of an exercise regimen or a rehabilitation plan.
   31. The method for determining patient rehabilitation protocol of any of examples 26 to 30, wherein the clinical input comprises at least one of time since surgery or time in rehabilitation.
   32. The method for determining patient rehabilitation protocol of any of examples 26 to 31, wherein the patient input is processed in real time from the data hub.

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

What is claimed is:

1. A patient brace system, comprising:
a crutch;
at least one crutch sensor configured to measure one or more parameters associated with the crutch and generate crutch signals in view of the same;
a controller operable to receive the generated crutch signals from the at least one crutch sensor, and further operable to process the generated crutch signals to produce patient gait data and communicate at least one of the one or more parameters measured by the at least one crutch sensor or the patient gait data to a data hub;
a patient brace; and
a rehabilitation application in communication with the data hub and configured to receive a clinical input, the rehabilitation application being configured to process the clinical input and the patient gait data to determine a response output and communicate the response output to at least one of a caregiver, a user of the patient brace, or to the patient brace.

2. The patient brace system of claim 1, wherein the system further comprises:
at least one brace sensor configured to measure one or more parameters associated with the brace and generate brace signals in view of the same; and
a second controller operable to receive the generated brace signals from the at least one brace sensor, and further operable to process the generated brace signals to produce patient brace data and communicate at least one of the one or more parameters measured by the at least one brace sensor or the patient brace data to the data hub,
wherein the rehabilitation application is configured to process the patient brace data in conjunction with determining the response output.

3. The patient brace system of claim 2,
wherein the at least one crutch sensor and the controller is located on the crutch,
wherein the at least one brace sensor and the second controller is located on the brace, and
wherein the controller and the second controller are configured to transmit the patient gait data and the patient brace data, respectively, to each other.

4. The patient brace system of claim 2, wherein the at least one brace sensor is operable to determine at least one of movement or location of the patient brace in relation to the crutch.

5. The patient brace system of claim 2, wherein the patient brace data comprises at least one of: knee angle, knee location, patient posture, muscle tone, swing time, or toe touch force data.

6. The patient brace system of claim 2,
wherein the second controller is operable to receive the output response generated by the rehabilitation application, and is further operable to process the generated output response, and
wherein the brace responds to the output response.

7. The patient brace system of claim 6, wherein the brace includes at least one of at least one actuator or at least one electrode configured to stimulate at least one of a patient muscle, change patient brace stiffness, or change freedom of patient brace movement.

8. The patient brace system of claim 7, wherein the response output is passive and includes at least one of an audible indication, a visual indication, or a tactile indication to a user.

9. The patient brace system of claim 7, wherein the response output is active and occurs in real time.

10. The patient brace system of claim 1, further comprising:
a second controller operable to receive at least one of the one or more parameters measured by the at least one crutch sensor or the patient gait data, and is further operable to process at least one of the one or more parameters measured by the at least one crutch sensor or the patient gait data and determine a load acting on at least one of the patient brace or patient knee.

11. The patient brace system of claim 1, wherein the crutch is a first crutch, and the patient brace system further comprises a second crutch used as a standard known datum for calibration of patient gait data.

12. A method of adjusting a knee brace, comprising:
determining patient gait data based on one or more parameters associated with a crutch;
communicating the determined patient gait data to at least one of a controller or a data hub; and
based on the communicated patient gait data, performing at least one of the following actions:
causing a knee brace to make an auto adjustment in response to the communicated patient gait data; or
instructing at least one of a caregiver or a user of a knee brace to adjust the knee brace.

13. The method of adjusting the knee brace of claim 12, wherein the controller is located on a knee brace, and wherein communicating the gait data depends on a positional relationship between the knee brace and the crutch.

14. The method of adjusting the knee brace of claim 12,
wherein at least one crutch sensor is configured to measure the one or more crutch parameters associated with the patient data gait, and
wherein communicating the determined patient gait data to at least one of a controller or a data hub further comprises operating the controller to process the one or more crutch parameters to produce the patient gait data.

15. The method of adjusting the knee brace of claim 12, further comprising operating a second controller to process one or more brace parameters measured by at least one brace sensor on the knee brace to produce patient brace data.

16. The method of adjusting the knee brace of claim 12, wherein causing the knee brace to make the auto adjustment further comprises operating at least one of at least one brace actuator or at least one electrode based on the patient gait data.

17. The method of adjusting the knee brace of claim 12, wherein causing the knee brace to make the auto adjustment further comprises using the patient gait data and the patient brace data in a load balancing algorithm.

18. The method of adjusting the knee brace of claim 12, wherein causing the knee brace to make the auto adjustment further comprises operating at least one of the at least one brace actuator or the at least one electrode to change at least one of a knee brace stiffness or a knee brace freedom of motion.

19. The method of adjusting the knee brace of claim 12, wherein causing the knee brace to make the auto adjustment further comprises transferring energy between the crutch and the knee brace.

20. A method for formulating a patient rehabilitation protocol comprising:
processing patient input from a data hub;
processing clinical input from a caregiver;
assessing patient gait based on the patient input and the clinical input by using at least one of inverse kinematics or inverse dynamics; and
at least one of providing or adjusting a patient rehabilitation protocol based on the patient gait,
wherein the patient input comprises at least one of patient gait data or patient brace data.

21. The method for determining patient rehabilitation protocol of claim 20,
wherein the patient rehabilitation protocol comprises a sensory guide, and
wherein the method further comprises operating the sensory guide to determine which muscle or muscles need stimulation.
